(19) United States
(12) Patent Application Publication
(10) Pub. No.: US 2021/0198623 A1
(43) Pub. Date: Jul. 1, 2021

(54) MACROCARRIER

(71) Applicant: Oxford University Innovation Limited, Oxford (GB)

(72) Inventors: Linh NGUYEN THUY BA, Oxford (Oxfordshire) (GB); Hua YE, Oxford (Oxfordshire) (GB); Zhanfeng CUI, Oxford (Oxfordshire) (GB)

(21) Appl. No.: 16/761,825

(22) PCT Filed: Nov. 9, 2018
(86) PCT No.: PCT/GB2018/053249

§ 371 Date: May 6, 2020

(30) Foreign Application Priority Data
Nov. 9, 2017 (GB) 1718556.2

(51) Int. Cl. C12N 5/00; C12M 1/12; C12N 5/0775

(52) U.S. Cl. CPC C12N 5/0068; C12M 25/18; C12N 2539/10; C12N 2533/30; C12N 5/0663

(57) ABSTRACT

A macrocarrier for the propagation of biological cells is described. The macrocarrier comprises substrate particles that are coated with a thermoresponsive polymer, which is capable of providing the macrocarrier with a cell-receiving surface and responding to a change in temperature to release cells from the macrocarrier. At least 50% of the substrate particles have a particle size of at least 1 mm. A system for the propagation of biological cells and a process for the propagation of biological cells are also described.

The present disclosure relates to a macrocarrier for the propagation of biological cells. The present disclosure also relates to system for the propagation of biological cells, as well as to a process for the propagation of biological cells.

### BACKGROUND

The industrial production of vaccines, enzymes, hormones and cytokines requires cells to be produced on a significant scale. Furthermore, recent advances in stem cell therapy and other cell-based therapeutic treatments often require a scalable quantity of cells to be produced.

Cells may be propagated in a bioreactor, where cells are grown on suspended microcarriers in a culture medium. The microcarriers act as supporting substrates to which cells are anchored during cell culturing process. Microcarriers are relatively small and typically range from 125 to 250 μm in size. Accordingly, they are easily suspended in culturing media and have a relatively high surface area to volume ratio for supporting cell attachment and growth. Following the culture stage, the anchored cells require separation from the microcarrier beads in order to be recovered.

There are a range of methods for recovering cells from microcarriers. For example, the cells may be recovered by enzymatic digestion, for example, using trypsin, accutase or collagenase. However, while such methods may be effective for separating the cells from the microcarriers, the treatment can sometimes have a negative effect on the physiology of the cells produced. This can have a negative effect on the quality and viability of the recovered cells.

Thermoresponsive polymers are polymers that show a significant change in properties upon a small change in temperature. An example of such a polymer is poly-N-isopropylacrylamide (PNIPAAm). Depending on the temperature, PNIPAAm can change from a hydrophilic, random coil conformation to a hydrophobic, collapsed globular conformation. Biological cells are typically attracted to the hydrophobic surface and repelled from the hydrophilic surface. Accordingly, the polymer can be used to provide a cell-receiving surface e.g. above a threshold temperature and a cell-releasing surface as the temperature falls. As such, cells may be anchored to the polymer above a threshold temperature, until such time as separation and isolation are required, whereby the temperature is lowered, and the cells detach. Unlike enzymatic treatments, this method of detachment may reduce the risk of damage to the physiology of the cell. In Cell Transplantation, Vol. 19, pp. 1123-1132, 2010, the thermosensitive polymer, PNIPAAm, is conjugated onto microcarrier beads having a diameter of 170 to 380 microns.

### BRIEF DESCRIPTION OF THE FIGURES

Aspects of the present disclosure are shown schematically, by way of example only, in the accompanying drawings, in which:

FIG. 1 is a schematic diagram of thermo-responsive polymer grafting onto the surfaces of PCL and the temperature-dependent effect of cell attachment to and detachment from the grafted surface;

FIGS. 2a and 2b show FTIR and XPS spectra showing the conjugation of PNIPAAm-NH₂ to the surface of PCL beads;

FIG. 3 shows cell proliferation on macrocarrier surfaces;

FIGS. 4a and 4b show cell detachment and cell viability data of cells detached from macrocarrier surfaces (i.e. trypsinization vs. reduced temperature comparison of cell detachment ratio and viability);

FIG. 5 shows recovered cell proliferation comparisons between different cells detached by reduced temperature and trypsinization;

FIG. 6 is a Western blot analysis of proteins collected from cells grown on tissue culture plates and thermoresponsive macrocarriers; the cells were detached by trypsin-EDTA and by reducing the temperature;

FIG. 7 is a diagram containing a series of images that show the preparation of PCL pellets;

FIG. 8 shows SEM images of PCL beads that were prepared according to the method described in Example 2 and SEM images of PCL beads that are commercially available;

FIG. 9 shows an SEM image of PCL beads and a series of histograms showing the size distribution of the beads;

FIG. 10 show FTIR and XPS spectra of PCL beads that were prepared according to the method described in Example 2 and PCL beads that are commercially available;

FIG. 11 shows SEM images and EDS spectra of porous PCL beads and PCL-PNIPAAm macrocarriers;

FIG. 12 shows an SEM image of a pore on the surface of a PCL-PNIPAAm macrocarrier;

FIG. 13 is a series of images showing the cell proliferation of MSC seeded on PCL and PCL-PNIPAAm macrocarriers as observed by a fluorescence microscope in low magnification;

FIG. 14 is a series of images showing the cell proliferation of MSC seeded on PCL and PCL-PNIPAAm macrocarriers as observed by a fluorescence microscope in high magnification;

FIGS. 15 and 16 each show histograms that illustrate the cell proliferation on various macrocarrier surfaces over several days; and

FIG. 17 shows images of MSC detached from PCL-PNIPAAm.

### DETAILED DESCRIPTION

In one aspect of the present invention, there is provided a macrocarrier for the propagation of biological cells. The macrocarrier comprises substrate particles that are coated with a thermoresponsive polymer that is capable of providing the macrocarrier with a cell-receiving surface and responding to a change in temperature to release cells from the macrocarrier, wherein at least 50% of the substrate particles have a particle size of at least 1 mm.

In another aspect, there is provided a system for the propagation of biological cells. The system comprises a bio-reactor and a macrocarrier as described in the present disclosure.

In yet another aspect, there is provided a process for the propagation of biological cells. The process comprises contacting biological cells with a macrocarrier of the present disclosure in a cell culturing medium; subjecting the macrocarrier to a temperature at which the thermoresponsive polymer presents a cell-receiving surface and propagating the cells on the macrocarrier; and subsequently altering the temperature of the macrocarrier to release any propagated cells from the macrocarrier.

Advantageously, a significant proportion of the substrate particles have a particle size of at least 1 mm. In contrast to microcarriers employed in prior art bioreactor systems that are typically 125 to 250 μm in size, a larger particle size can present biological cells with a ‘flatter’ surface upon which to adhere. It is believed that this flatter surface results in the adhered cells being less torsional constrained, or twisted, and subjected to less shearing force or mechanical stress during agitation in the bioreactor. As a result, the cells may be exposed to a gentler, more uniform, and optimal growing environment. This can help to improve the quality (e.g. viability and health) of the cells produced. Moreover, because the macrocarriers of the present disclosure are coated with a thermoresponsive polymer, cells can be released from the macrocarrier by changing the surrounding temperature. This allows the cells to be recovered without the need, for example, of enzymatic treatments that can present an increased risk of cell damage.

The larger particle size may also aid cell collection and separation from the macrocarrier when compared to a microcarrier. It is thought that a cell will require more energy to detach from a microcarrier in comparison to a macrocarrier, due to differences in the radii and surface area curvatures of the carriers.

On microcarriers, cells tend to clump and grow in aggregate when they are cultured in a bioreactor. In many clinical, biotechnological and tissue engineering settings, it is necessary to produce discrete cells and the production of clumps of cells can be problematic. When cells are cultured on macrocarriers in a bioreactor they do not, in general, aggregate. Cells attached to a macrocarrier are also less likely to suffer damage in a bioreactor compared to cells attached to a microcarrier.

As noted above, at least 50% of the substrate particles have a particle size of at least 1 mm. In some examples, at least 70%, preferably at least 80 or 90% of the substrate particles have a particle size of at least 1 mm. By ensuring that a significant proportion of the substrate particles are large, advantage can be taken of the “flatter” support surface. As described above, this allows the cells to be cultivated under gentler conditions, reducing the shear forces to which they are exposed during propagation within the bioreactor. This, in turn, can result in e.g. healthier and more viable cells.

It is preferred that the substrate particles have a relatively narrow size distribution. For example, the substrate particles may be sufficiently large to present a desirable surface for cell attachment and propagation but, at the same time, be sufficiently small to be suspended in the culture medium of a bioreactor. For instance, at least 50% of the substrate particles may have a particle size of 1 mm to 10 mm. In other examples, at least 50% of the substrate particles have a particle size of 2 mm to 6 mm. In other examples, at least 50% of the substrate particles have a particle size of 3 mm to 5 mm. In other examples, at least 70% of the substrate particles have a particle size of 1 mm to 10 mm. In other examples, at least 70% of the substrate particles have a particle size of 2 mm to 6 mm. In other examples, at least 70% of the substrate particles have a particle size of 3 mm to 5 mm. In other examples, at least 80% or 90% of the substrate particles have a particle size of 1 mm to 10 mm. In other examples, at least 80% or 90% of the substrate particles have a particle size of 2 mm to 6 mm. In other examples, at least 80% or 90% of the substrate particles have a particle size of 3 mm to 5 mm. In a preferred embodiment, 95 to 100% of the substrate particles have a particle size of 1 to 10 mm, preferably 2 to 6 mm, and more preferably 3 to 5 mm.

Any suitable substrate particle may be used in the macrocarrier of the present disclosure. For example, the substrate particle may be of any suitable shape, including, for example, a substantially spherical, ellipsoid, ovoid, or ring shape. In one example, the substrate particle may take the form of a bead. The bead may be of any suitable shape, for example, substantially spherical, elliptical, ovoid, or ring. Where the substrate particles are non-spherical, their particle size may refer to the largest linear dimension across the substrate particle. For example, where the substrate particles are ellipsoid, the particle size may refer to the length of the major axis of the ellipsoid. It may be preferable that the substrate particle(s) is/are substantially spherical.

The substrate particle may be formed of any suitable material. Preferably, the material is a biocompatible material, for example, a biocompatible polymer. In some examples, the material is biocompatible according to ISO 10993. Suitable materials include polysaccharide, protein, glass, polystyrene, polyester, polyolefin, silica, silicone, polyacrylamide and polyacrylate. Suitable polysaccharides include dextran (e.g. diethylaminoethanol (DEAE)-dextran), chitosan and alginate. A suitable protein may be collagen. An example of a suitable polyacrylate is poly(2-hydroxyethyl methacrylate). Preferably, the substrate particle comprises a polyester.

In a preferred embodiment, the substrate particle comprises a polyester selected from the group consisting of polylactic acid (“PLA”), polyglycolic acid (“PGA”), polycaprolactone (“PCL”), polybutyrolactone (“PBL”), polyvalerolactone (“PVL”), polyhydroxybutyrate (“PHB”), poly (3-hydroxy valerate), poly(ethylene succinate) (“PESu”), and poly(butylene succinate) (“PBSu”). In a more preferred embodiment, the polyester is PCL.

Any suitable thermoresponsive polymer may be used in the macrocarrier of the present disclosure. A thermoresponsive polymer, also referred to as a thermosensitive polymer, is a polymer that shows a significant change in properties upon a small change in temperature. In the present disclosure, the thermoresponsive polymer that is used to coat the substrate particles is capable of providing the macrocarrier with a cell-receiving surface onto which cells can be attached. The thermoresponsive polymer, however, responds to a change in temperature to release cells from the macrocarrier.

In some examples, the thermoresponsive polymer is one that changes its morphology upon exposure to a change in temperature. The thermoresponsive polymer may be made up of hydrophobic and hydrophilic parts that change their orientation upon a change in temperature, so as to present a hydrophobic surface or a hydrophilic surface to the external environment. For instance, in one embodiment, the thermoresponsive polymer changes from a hydrophilic, random coil conformation to a hydrophobic, collapsed globular conformation in response to a change in temperature. The hydrophobic surface may be presented to allow, for example, immobilisation or attachment of cells. On the other hand, the hydrophilic surface may repel the attached cells, allowing them to be released from the polymer surface.

In some examples, the threshold temperature is at or below a temperature suitable for culturing, propagating, or differentiating cells. In another example, the threshold temperature is above a temperature suitable for detaching cells but maintaining quality and viability of the cells. In another example, the temperature range difference between the thermoresponsive polymer providing a cell-receiving surface and a cell-repelling surface is optimised so as to maximise cell tethering for culturing, propagation or differentiation, but maximise cell detaching once the culturing, propagation or differentiation step is complete. In another example, the difference in temperature between that at which cells tether and that at which cells detach should be minimised so as to reduce the possibility of cold shock, or low-temperature stress, on the propagated cells.

In some examples, the thermoresponsive polymer provides the macrocarrier with a cell-receiving (e.g. hydrophobic) surface above a threshold temperature and releases cells (e.g. by presenting a hydrophilic surface) from the macrocarrier below the threshold temperature. Accordingly, the macrocarrier may be maintained above the threshold temperature (e.g. in a bioreactor) to allow attachment of cells and facilitate their propagation. Subsequently, the temperature may be reduced below the threshold temperature to release the propagated cells from the macrocarrier. This can allow convenient cell recovery.

In some examples, the threshold temperature may be a temperature of between 20° C. and 40° C. In some examples, the threshold temperature is a temperature of between 30° C. and 40° C. In some examples, the threshold temperature is a temperature of between 30° C. to 37° C. In some examples, the threshold temperature is about 32° C.

In some examples, the macrocarrier may be maintained above the threshold temperature at a temperature that facilitates or optimises cell propagation and growth in order to allow for cell attachment and propagation on the macrocarrier. This temperature may be, for example, about 34 to 39° C., preferably, at about 37° C. Subsequently, the macrocarrier may be cooled, for instance, to below the threshold temperature of the thermoresponsive polymer to release the cells. This macrocarrier may be cooled to a temperature below 33° C., for instance, below 32° C. In some examples, the macrocarrier may be cooled to 30° C. to release the propagated cells.

A number of thermoresponsive polymers may be used to coat the macrocarrier, such as a polymer selected from the group consisting of poly(N-isopropyl acrylamide) (“PNIPAAm”), poly(butylmethacrylate) (“PBMA”), poly(D,L-lactide) (“PDDLA”), poly(N,N-diethylacrylamide) (“PDEAAm”), poly(N-vinylcaprolactam) (“PNVCL”), poly[2-(dimethylamino)ethyl methacrylate] (“PDMAEMA”), poly(ethylene oxide-b-propylene oxide-b-ethylene oxide) (“PEO-PPO-PEO”), poly(ethylene glycol-b-(DL-lactic acid-co-glycolic acid)-b-ethylene glycol) (“PEG-PLGA-PEG”), poly(methyl 2-propionamidoacrylate) (“PMPA”), poly([DL-lactic acid-co-glycolic acid]-b-ethylene glycol-b-[DL-lactic acid-co-glycolic acid]) (“PLGA-PEG-PLGA”). In an example, the thermoresponsive polymer is PNIPAAm. Depending on the temperature, PNIPAAm can change from a hydrophilic, random coil conformation to a hydrophobic, collapsed globular conformation.

The thermoresponsive polymer may be coated onto the substrate particles using any suitable method. The substrate particles may be at least partially coated with the thermoresponsive polymer.

In some examples, the thermoresponsive polymer may be covalently attached to the substrate particle. The thermoresponsive polymer may be suitably functionalised with a group capable of forming a covalent bond to a suitably functionalised substrate particle. In some examples, the thermoresponsive polymer may be functionalised with more than one type of functional group. In some examples, the substrate particle may be functionalised with more than one type of functional group. In some examples, more than one thermoresponsive polymer is attached to one substrate particle. In some examples, the thermoresponsive polymer is functionalised with an amino group and the substrate particle is functionalised with an acid group, thereby allowing the formation of an amide bond as the covalent linkage. In another example, the thermoresponsive polymer could be functionalised with the acid group and the substrate particle functionalised with the amino group, thereby allowing the reverse amide bond to be formed as the covalent linkage. In some examples, the thermoresponsive polymer is PNIPAAm that is functionalised with an amino group, and the substrate particle is PCL that is functionalised with an acid group. Other complementary groups on PCL and PNIPAAm capable of forming amide bonds, such as for example esters, acyl halides, and anhydrides, are encompassed within the scope of the disclosure, as are complementary groups capable of forming covalent bonds other than amide bonds. Alternative complementary functional groups for coupling the substrate particle to the thermosensitive polymer will be apparent to the skilled person as means to covalently link these moieties other than through an amide bond. Examples of such coupling groups include hydroxyl, thiol, halo, sulphonyl, aldehyde, epoxy, and the like.

Typically, the substrate particle does not comprise a thermoresponsive polymer. Thus, the substrate particle may not be made of a thermoresponsive polymer.

The substrate particles may be solid or porous. Where porous particles are used, oxygen in the culture medium may diffuse through the particles towards any cells attached to the particles surface. Porous particles may have a lower density than non-porous particles made of the same material that occupy the same volume. When the substrate particles have a relatively low density, the resulting macrocarriers may float in the bioreactor or may be easily stirred or swirled within the bioreactor.

The substrate particles may have a relative density with respect to water of less than 1 (i.e. at atmospheric pressure and a temperature of 20° C.), such as <0.85, particularly <0.70.

The bulk density of the substrate particles is typically less than the true density of the solid material from which the substrate particles is made. The term “bulk density” as used herein refers to the mass of one or more substrate particles divided by the total volume that they occupy. The bulk density measurement includes the volume of the solid material from which the particles are made and any pores, whether open or closed. The term “true density” as used herein refers to the density of the solid material from which the particles are made. The measurement of true density therefore excludes the volume of any open and closed pores.

The substrate particles may have a ratio of bulk density to true density of <0.80:1, such as <0.70:1, particularly <0.60:1. For the avoidance of doubt, the bulk density and true density measurements should be determined at atmospheric pressure and a temperature of 20° C.

The surfaces of the substrate particles may be porous. The porous surfaces of the substrate particles may be retained after coating with a thermoresponsive polymer.

The macrocarrier of the present disclosure may have a surface, such as the cell-receiving surface, that is porous.

The pores on the surface of the macrocarrier can absorb and retain both nutrients and medium, which are needed for cell growth. The close proximity of nutrients and medium may facilitate the rapid propagation of cells, such that a significant number of high quality cells can be produced over a shorter time. For example, the stationary phase for cell growth may be reached more quickly using macrocarriers having a porous surface compared to macrocarriers having a non-porous and dense surface.

In comparison to macrocarriers having a non-porous surface, the surface of porous macrocarriers is relatively rough. This relatively rough surface can aid cell proliferation because cells adhere to rough surfaces better than they adhere to smooth surfaces.

When a surface of the macrocarrier, such as the cell-receiving surface, is porous, then the pores may have a size of ≤20 μm, such as ≤10 μm, particularly ≤5 μm. The pores typically have a size that is less than the size of a grown cell. Pore size may be determined by SEM imaging.

The macrocarrier of the present disclosure may be used to propagate any biological cell. In other words, the cells may be any cell capable of adhering and growing on the surface of the macrocarrier. Examples include mammalian as well as hybrid cell lines and tumour-based cells. Preferably, the cells are mammalian cells. The mammalian cells may be cultured for the following tissue types: for example, bone marrow, carcinoma, conjunctiva, cornea, endothelium, epithelium, fibroblast, fibrosarcoma, heart, hepatoma, liver, lung, macrophage, melanoma, muscle, neuroblastoma, osteosarcoma, ovary, pancreas, pituitary, rhabdomyosarcoma, synovial fluid, thyroid, and the like.

As an example, the following cell types may be propagated on macrocarriers of the present disclosure: bovine (endothelial, kidney, muscle), canine (MDCK), chicken (embryo, fibroblast, muscle, myoblasts), fish (RTG-2, AS, CHSE-214), guinea pig (GPK), hamster (BHK, BHK21, BHK21 C13, CHO, CHO-K1, CHO-recombinant)), human (adenocarcinoma, amniotic, bladder cancer, breast cancer, endothelial, fibroblast, FS, FS-4, HeLa, HEL 299, IMR, K-562, KB, kidney, lymphoblastoid, lymphocyte, MCF-7, monocyte, MRC-5, osteosarcoma, pancreas), monkey (BSC-1, CV-1, kidney, LLC-MK, Vero), mouse (fibroblast, L-929, macrophage, mesenchyme), pig (endothelial, testicular, thyroid), rat (epithelium, myoblast, pancreas, pituitary), and turkey (pituitary). In addition, cells that are cultures on macrocarriers can be used as substrates for the production of vaccines, vectors, natural and recombinant proteins, monoclonal antibodies, and other biological products.

In another example, a composition is provided of a plurality of cells tethered to a macrocarrier. Some examples are mesenchymal stem cells (“MSCs”), human dermal fibroblasts (“HDFs”), human umbilical vein endothelial cells (HUVEC) and neuroblastoma Sy5y cells.

In another example, a system is provided for biochemical engineering, wherein the proliferated cells remain tethered to the macrocarrier for further culturing so as to generate a variety of downstream products, prior to cell release. Such downstream products could include for example cell therapy products from bioprocessed stem cells, recombinant protein production, antibody and virus generation, and gene amplification, to name a few possible biomanufacturing applications. Conditions for fibroblast cell culture systems for synthesizing extracellular matrix and collagen could be utilised in the tethered fibroblast macrocarrier system herein.

### EXAMPLES

### Example 1

### Materials and Methods

### Materials

All materials were purchased from Sigma-Aldrich (UK) and used as received. The materials were: polycaprolactone pellets (PCL, Mn 80,000), sodium hydroxide (NaOH), 1-ethyl-3-[3-dimethylaminopropyl]carbodiimide hydrochloride (EDC), Sulfo-N-hydroxysuccinimide (Sulfo-NHS), morpholinoethanesulphonic acid (MES) and poly (N-isopropylacrylamide) amine terminated average Mn 2500 (T) (PNIPAAm-NH₂). Deionised water (DI water) used in this study was obtained from an ultrapure water purification system (Elix®, Millipore).

### Preparation of PCL-PNIPAAm

The PCL pellets were immersed in NaOH 1M solution for 1 h with constant shaking to obtain carboxylate ions PCL-OOO⁻, then they were rinsed with for autoclaved deionized water (DI) several times. PCL-PNIPAAm macro-carriers were synthesized by conjugating PCL-OOO⁻ pellets with PNIPAAm-NH₂ through an amidation reaction. The PCL-OOO⁻ pellets were activated by 1-ethyl-3-[3-dimethylaminopropyl]carbodiimide hydrochloride (EDC, 0.12M) and Sulfo-N-hydroxysuccinimide (Sulfo-NHS, 0.06M) in 0.05M morpholinoethanesulphonic acid (MES, 0.05M) buffer solution (pH 6) for 3 h at room temperature. PNIPAAm-NH₂ was added to the activated PCL-COO⁻ solution and gently shaken at 4° C. overnight. The solution containing PCL-PNIPAAm macro-carriers was centrifuged at 1500 rpm for 10 min, washed five times with deionized distilled water, and lyophilized for 2 days.

The reaction scheme is shown schematically below:

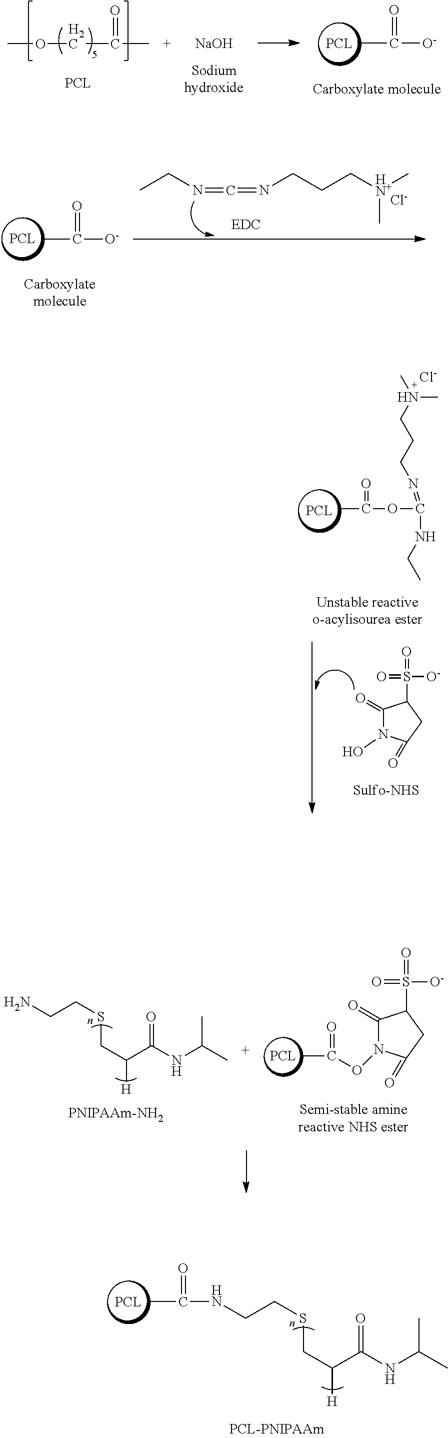

### Characterizations and Measurements

Fourier transform infrared (FTIR) spectra were recorded using an FTIR spectrometer (Bruker, Tensor 27) equipped with attenuated total reflectance (ATR, Pike). Before collecting sample spectra, the background spectrum was collected by measuring the response of the spectrometer without a sample.

X-ray photoelectron spectroscopy (XPS) spectra for PCL and PCL-PNIPAAm were obtained base pressure 1×10⁻⁹ torr, variable aperture 3-10 mm and data analysed using CasaXPS peak fitting software.

### Cell Culture on Thermo-Responsive Macrocarriers

Human dermal fibroblast cells (HDF, ThermoFisher Scientific) were cultured in Dulbecco's modified Eagle's medium (DMEM 4.5 mg/l of glucose; Gibco BRL, Gaithersburg, Md. USA) supplemented with 10% (v/v) fetal bovine serum (FBS; Gibco BRL) and 1% (v/v) penicillin-streptomycin (PS; Gibco BRL) and Green Fluorescence Protein (GFP) was cloned into Mesenchymal stem cells (MSC, kindly provided from Department of Paediatrics and Adolescent Medicine, LKS Faculty of Medicine, The University of Hong Kong). Siliconized (Sigmacote® treated) glass bottles were prepared prior to use to prevent cells adhering to the bottle walls. PCL and PCL-PNIPAAm macrocarriers were washed with phosphate buffer saline (PBS) for 15 min and incubated in DMEM at 37° C., overnight.

### Cell Viability, Cytotoxicity and Proliferation Assessment

Cell viability, cytotoxicity and proliferation were determined by CCK-8 assay (Sigma). To measure the efficiency of cell attachment to macrocarriers within 24 h of culture, the macrocarrier-free supernatant was carefully removed and the number of cells in the supernatant was determined with a hemocytometer. The number of cells attached to macrocarriers was calculated by subtracting the number of cells in the supernatant from the total cell number at inoculation. The attachment yield was calculated as follows:

Attachment yield (%)=(number of cells attached to macrocarriers/total number of cells number at inoculation)×100.

Cell Detachment from Macrocarriers

After 1 day of suspension culture, the temperature of the culture medium was reduced from 37° C. to 30° C. by using an incubator and HDF cultured on two types of macrocarriers were incubated for 40 min at 30° C. The number of detached cells in the macrocarrier-free supernatant was counted with a hemocytometer.

Cell detachment ratio=[number of detached cells]/[total number of attached cells on macro-carriers before detachment]×100.

### Extra Cellular Matrix (ECM) Protein Expression

ECM protein expression of HDF was analysed by western blot analysis. To detect the fibronectin, laminin and collagen I on the detached cells, anti-fibronectin (1:200, Santa Cruz, Biotechnology, Calif., USA), anti-laminin (1:200, Santa Cruz, Biotechnology, Calif., USA) and anti-collagen I (1:50, Santa Cruz, Biotechnology, Calif., USA) anti-bodies were used.

For western blot analysis: HDF were rinsed and harvested in lysis buffer (RIPA, Millipore, Milford, Mass., USA), vortexed on ice 5 times within 20 min and centrifuged for 10 min at 13,000 rpm at 4° C. Anti-fibronectin, anti-laminin and anti-collagen type I antibodies were used as proteins for the analysis. Anti-beta actin antibody was used as housekeeping control. The immunocomplexes were visualized using enhanced chemiluminescence reagent.

### Statistical Analysis

All quantitative data were expressed as mean±SD. Statistical analysis was performed with two-way analysis of variance (ANOVA) with Tukey's honestly significant difference post hoc test. All analyses were carried out using GraphPad Prism 6 with a value of p<0.05 was considered statistically significant.

### Results and Discussion

### Characterization of PNIPAAm Grafted PCL Macrocarriers

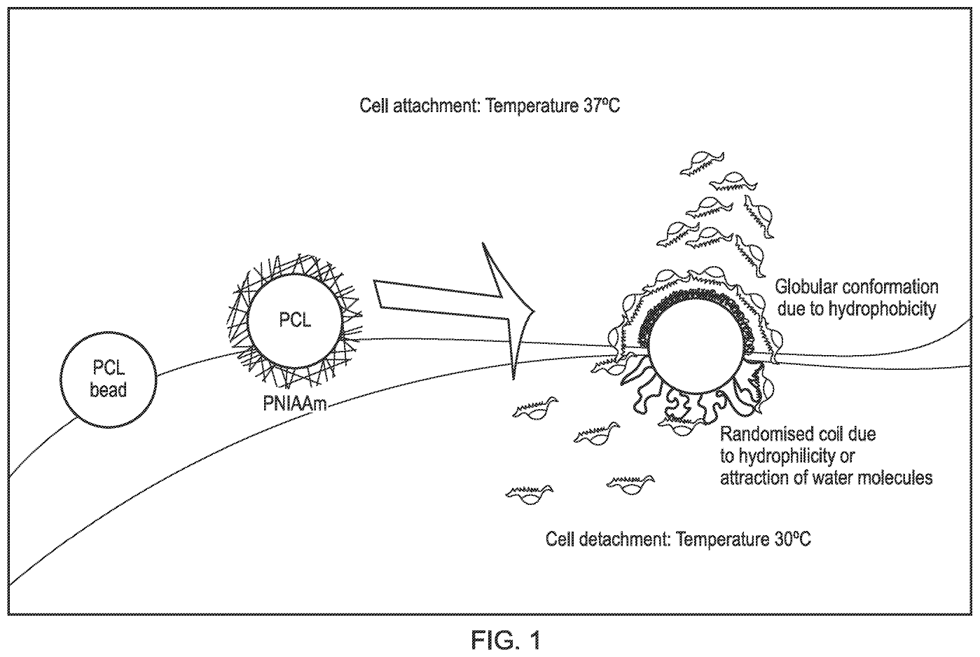

FIG. 1 shows a schematic diagram of thermo-responsive polymer grafting onto the surfaces of PCL and the temperature-dependent effect of cell attachment to and detachment from the grafted surface. Lowering the temperature as low as 30° C. cause cellular detachment due to the hydrophilic conformation of PNIPAAm. At 37° C. the conformation of PNIPAAm is globular conformation which provides a hydrophobic surface on the PCL macrocarrier. When the temperature is dropped down to 30° C., the conformation changes to randomised coil causing the surface to become hydrophilic in nature. A hydrophobic surface attracts cells and a hydrophilic surface repels the cells.

In order to graft the thermoresponsive polymer onto the surfaces of PCL beads, PNIPAAm-NH₂ polymers were conjugated with PCL beads through amidation between the carboxylate molecule on PCL beads' surface and the amine end group of PNIPAAm-NH₂. PCL pellets were initially treated with NaOH, which cause the base hydrolysis of esters bonds in PCL creating carboxylate ions. This carboxyl functional groups on PCL beads can be activated by EDC and NHS to form succinimide esters, which in turn spontaneously react with the amine groups on the PNIPAAm-NH₂. The reaction of carboxylate with EDC creating an unstable reactive O-acylisourea ester. Sulfo-NHS is then added to stabilize the intermediate, which converts the unstable O-acylisourea into an amine-reactive NHS ester. This ester will react with the amino end of the PNIPAAm-NH₂ to form a stable covalent amide bond between the PCL beads and the PNIPAAm-NH₂. The scheme of conjugation of PNIPAAm-NH₂ to PCL is shown in FIG. 1.

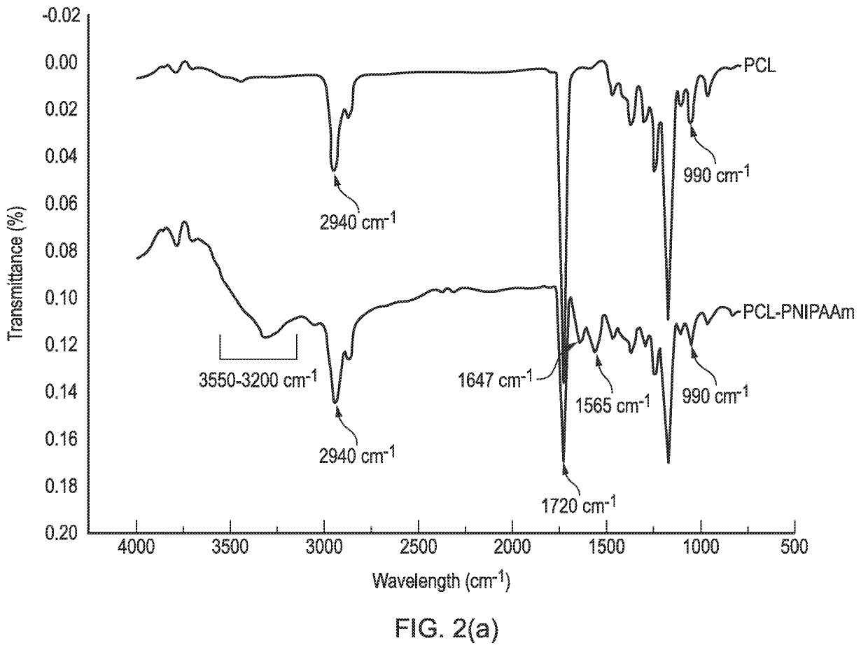

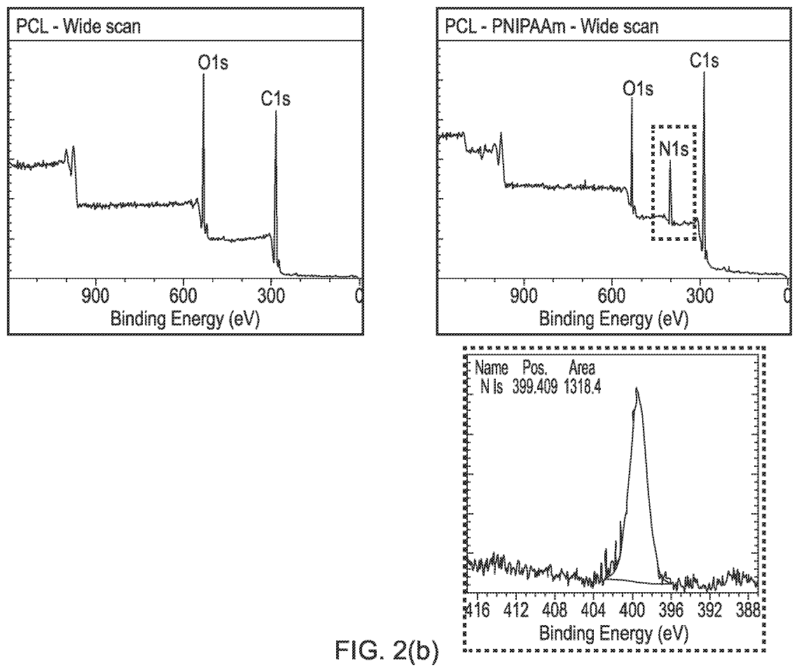

FTIR spectroscopy was used to confirm the conjugation of PNIPAAm-NH₂ to the surface of PCL beads. The FTIR spectrum in FIG. 2a shows the appearance of several new peaks due to PNIPAAm-NH₂ introduction onto PCL-COOH. The wide peak between 3550 and 3200 cm⁻¹ belongs to the N—H stretching of the modified PCL beads. The increasing of peak at 2940 cm⁻¹ is attributed to the vibration of aliphatic groups (—CH₂—)ₙ of the copolymer. The increasing of intensity peak at 1647 cm⁻¹ could be the sign of amide I bond, arising from C═O stretching and little C—N stretching of PNIPAAm. The peaks at 1565 cm⁻¹ corresponding to amide II bond, arising from N—H bending and C—N stretching of PNIPAAm. This suggested that the conjugation of the PNIPAAm on PCL beads was successful.

XPS determines the chemical composition of a surfaces top several nanometres. The appearance of a new N1s signal with binding energy at 400 eV after PNIPAAm grafting in the wide scan XPS spectra in FIG. 2b were indicative of successful grafting of PNIPAAm on the PCL macrocarriers surface. The N1s core-level spectra from PCL-P was curve-fitted with peak at 399.4 eV attributable to the amino group (—NH₂). Detection of N and C from C═O bonds means that PNIPAAm-NH₂ is present on the surface of PCL beads.

### Cell Viability, Cytotoxicity and Proliferation Assessment

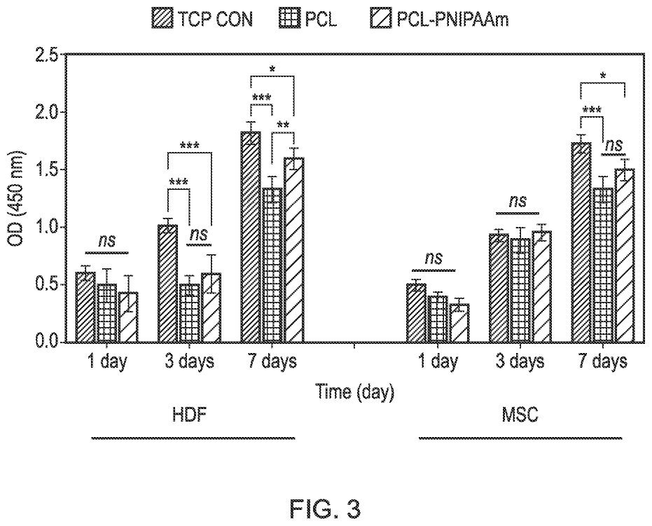

In order to assess whether the grafting material and other chemical reactions of the grafting procedure caused any adverse effect on cell growth and viability we used CCK-8 assay for viability up to 7 days. Although compared to the control both PCL and PCL-PNIPAAm surfaces showed reduced viability, a consistent increase in cell proliferation on both surfaces was depicted as shown in FIG. 3. The results showed that both HDF cells and MSCs survived and proliferated on both PCL and PCL-P (PCL-PNIPAAm) and tissue culture plate as control (TCP CON) surfaces for 1, 3 and 7 days (a). At day 7, a significant increase in OD was observed in both cell types compared to day 1 and 3 as well as a both cells survived higher on the surfaces of PCL-P than the surfaces of PCL (* p<0.05; ** p<0.01; *** p<0.001; ns: no significant different). In addition, a slightly healthier proliferation rate was observed in grafted surfaces than the non-grafted PCL (* p<0.05 at day 7).

Altogether these results suggested that PNIPAAm grafting onto PCL surfaces was risk-free and thus provided a valuable tool for recovering large-scale cellular collections.

Cell Detachment from Macrocarriers

In order to ascertain efficiency of cell detachment by lowering the temperature to 30° C. we compared the recovered cells in the reduced temperature condition with trypsinization conditions.

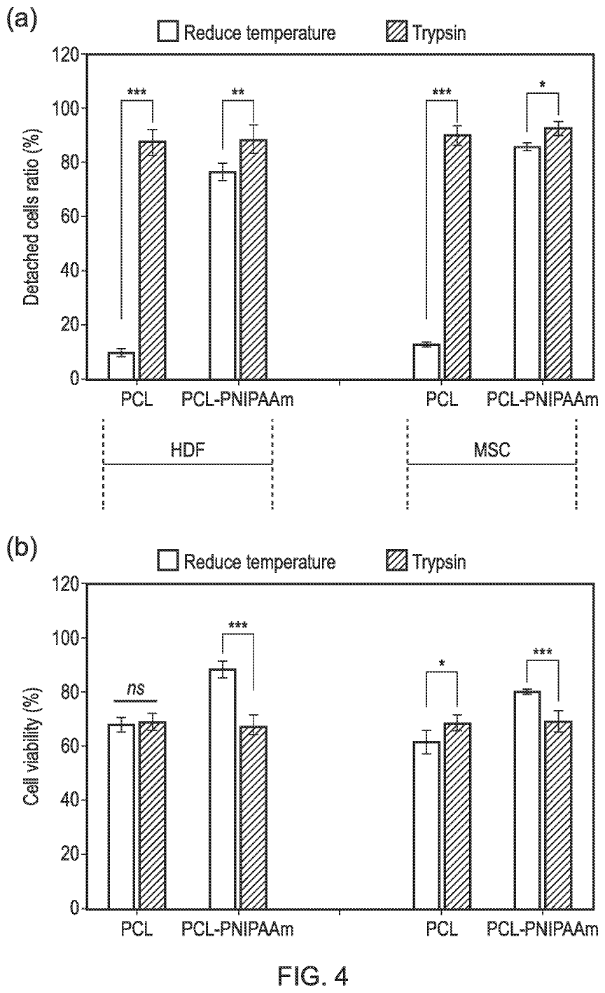

FIG. 4a shows that both HDF cells and MSCs in reduced temperature conditions (30° C.) showed a significantly higher cell-detachment ratio from PCL-PNIPAAm surfaces than from PCL only surfaces. Trypsinization did not show a significant difference in the extent of cell detachment between the surfaces but this technique showed more detachment of cells from the surfaces of both PCL and PCL-PNIPAAm surfaces. FIG. 4b shows that a higher cell viability rate was observed in temperature dependent cell recovery technique than by trypsinization. Both HDF and MSCs had higher survival when recovered from PCL-PNIPAAm than by trypsinization (* p<0.05; ** p<0.01; *** p<0.001; ns: no significant difference).

FIGS. 4a and 4b show that more than 70% of the cells were detached from PNIPAAm-grafted PCL surfaces by simply lowering the temperature. In contrast, trypsinization had a higher detachment rate than the reduced temperature technique of thermoresponsive polymer. This was not surprising; other research groups have also reported higher detachment rates with enzymatic digestion than with the thermal reduction technique. However, the physiological damage caused by trypsin or other enzymes is the major reason why researchers wish to avoid enzymatic digestion in clinical applications.

Recovered Cell Proliferation After Detachment from PCL-PNIPAAm

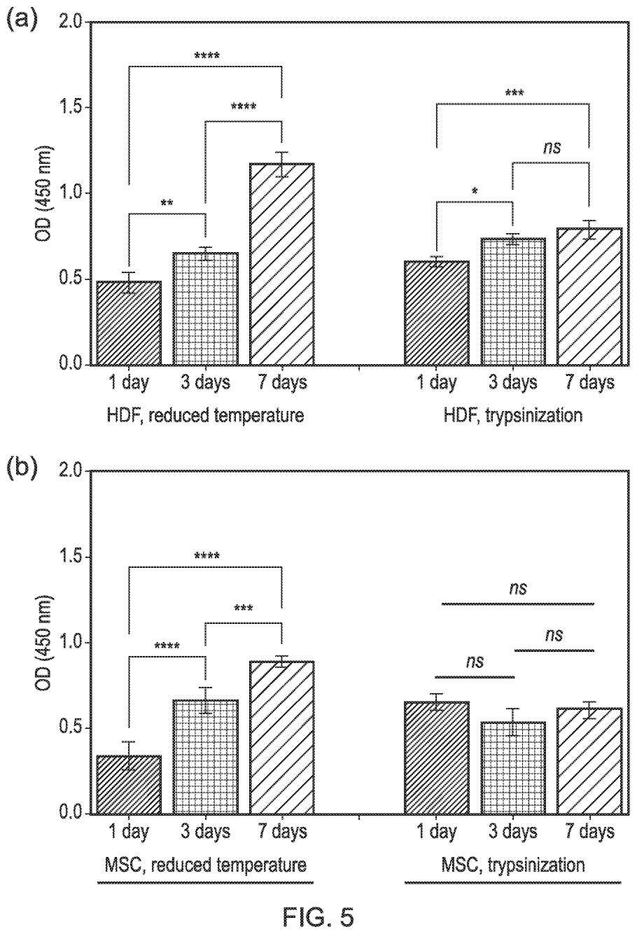

To demonstrate the propagation and proliferation into the immediate cellular passage of the harvested and recovered cells, a comparative viability assay was conducted for 1, 3 and 7 days between trypsin treated and recovered cells and reduced temperature and recovered cells (FIGS. 5a and 5b). Both HDF cells (FIG. 5a) and MSCs (FIG. 5b) from PCL-PNIPAAm surfaces were recovered, collected and grown at 1, 3 and 7 days in culture media. After recovering the cells either by using trypsin-EDTA or reduced temperature, equal numbers of cells were seeded for proliferations. CCK-8 studies showed when both cell types were recovered from PCL-PNIPAAm surfaces using reduced temperature, cell growths were exponential and significant over time. However, when collected by trypsinization, cell growths were non-exponential and insignificant over time (* p<0.05; ** p<0.01; *** p<0.001; **** p<0.0001; ns: no significant difference). The result showed a logarithmic growth with significant increase in cell numbers in both cell types collected from PCL-PNIPAAm surfaces by reducing temperature. This result clearly indicated that cell recovery process involving reduced temperature process was more efficient than the Trypsin-EDTA recovery process.

ECMs on Cells Detached from PCL-PNIPAAm Macrocarriers

The fate of the expression of ECM proteins on cells detached from PCL-PNIPAAm by either trypsin or reduces temperature was observed by immunoblotting. In this study, we seeded HDF on tissue culture plate and PCL-PNIPAAm, incubated for 7 days, and then collected them either trypsin-EDTA treatment or simply by reducing temperature. Cells were then immunostained or subjected for total protein collection for Western blotting. Three major structural ECM proteins such as Fibronectin (FN), Laminin (LM) and Collagen type I (Col I), which are the most abundant and important ECM proteins found in cells/tissues, play various roles in foetal development, tissue repair and angiogenesis. Because of their ‘glue-like’ properties, these proteins usually play a role in cell attachment on the surfaces. Here we studied these proteins to see whether the process employed to cell recovery affected their expression or not.

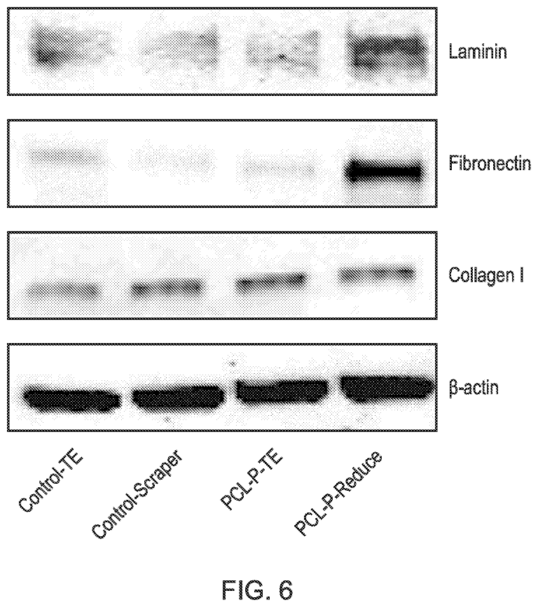

The expression patterns of Fibronectin, Laminin and Collagen I in recovered HDF cells (after detachment and collection) were analysed by Western blot as shown in FIG. 6. Total proteins were collected from cells grown in tissue culture plates by harvesting the total cells either using trypsin-EDTA (Control-TE) or by scraping the monolayer of cells by a scraper (Control-Scraper). Cells were also grown on PCL-PNIPAAm beads and harvested either by trypsin-EDTA (PCL-P-TE) by reducing the temperature (PCL-P-Reduce). Antigen against laminin, Fibronectin and Col 1 were used to detect the expression of these proteins by Western blot after harvesting these cells in four different ways. B-actin is the housekeeping protein.

The Fibronectin and Laminin expression in cells detached from PCL-PNIPAAm by reduced temperature was higher than the cells detached by trypsinization. However, Collagen 1 was found equally expressing in cells and non-significantly affected by the two different processes. The result was consistent with immunostaining data and indicated in a whole that trypsinization might adversely affect cell structure and physiology by simply degrading some of the structural ECM proteins as well.

### Example 2

### Materials and Methods

### Materials

Polycaprolactone pellets (PCL, Mn 80,000), polyvinyl alcohol (PVA, Mw=13-23 KDa, 87-89% hydrolysed) and dichloromethane (DCM) were purchased from Sigma-Aldrich (UK). Deionised water (DI water) used in this study was obtained from an ultrapure water purification system (EIix™, Millipore).

### Preparation of PCL Macro-Bead

PCL macro-beads were prepared using an established emulsion method, followed by the evaporation of the solvent used to liquefy the macrosphere polymer.

An aliquot of PCL pellets was dissolved in DCM to obtain 10, 12, 15 and 18 (w/v %) organic phases, while the PVA was dissolved into DI water to obtain 0.5, 1.0, 1.5, 2.0 and 3.0 (w/v %) inorganic phase.

A needle syringe (containing 5 ml of PCL solution) was placed on a pump which was used to form PCL/DCM solution droplets, which are precursors to the solidified beads. The 5 mL of formed PCL/DCM droplets were collected in a petri dish, containing 10 mL of PVA solution. The PVA solution was then decanted. DCM solvent was allowed to evaporate through the aqueous phase, thus resulting in droplet solidification, over 3 days in a fume hood.

### Results and Discussion

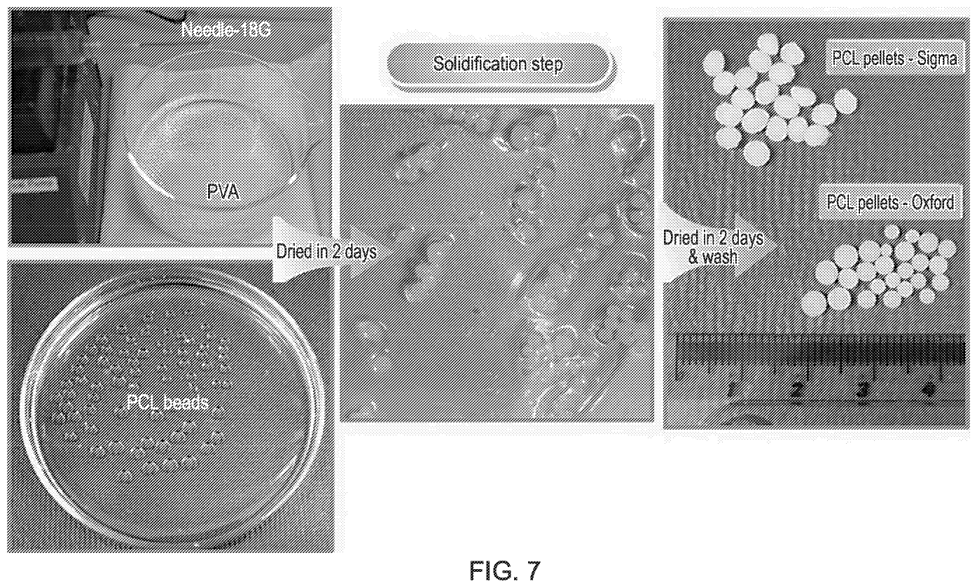

PCL beads were prepared by the o/w emulsion solvent evaporation process. In the first step, the organic phase was emulsified in the aqueous external phase. The organic phase was dichloromethane. Due to the low evaporation temperature of dichloromethane, the macrospheres formed faster than with other solvents having a higher evaporation temperature, such as chloroform. As the organic solvent evaporates from the surface of the droplets, the concentration of PCL increases and reaches a critical point at which the polymer concentration exceeds its solubility in the organic phase. At this critical point, it precipitates to produce macrospheres. The method used for fabrication of PCL macrospheres is shown in FIG. 7. The PCL pellets that were prepared from this method are also shown in FIG. 7 (see “PCL pellets—Oxford”) alongside the PCL pellets that were purchased from Sigma-Aldrich (“PCL pellets—Sigma”).

### Effect of Concentration of PCL on the Forming of the Beads

The influence of PCL concentration on the forming of the beads was probed using 10, 12, 15 and 18 wt/v%, as shown in Table 1.

TABLE 1
Effect of PCL concentration on bead formation

| PCL | | Formed the | Rate of | PVA |
|---|---|---|---|---|
| concentration | | beads (after | pump | concentration |
| (wt/v %) | Droplet | solidification) | (ml/min) | (wt/v %) |
| 10 | Yes | No | 0.4 | 3 |
| 12 | Yes | No | | |
| 15 | Yes | Yes | | |
| 18 | No | N/A | | |

Droplets were formed at a PCL concentration of 10, 12 and 15 wt/v% while there was no droplet formation at 18 wt/v%. After the solidification step, the beads were obtained from only 15% wt/v of PCL. Thus, the concentration of PCL at 15 wt/v% was chosen as the optimal concentration for further experiments.

### Effect of Flow Rates on Bead Size

Table 2 shows the relationship between the flow rate and the size of the beads.

TABLE 2
Effect of flow rates on bead size

| PCL | PVA | Rate of | Size of |
|---|---|---|---|
| concentration | concentration | pump | beads |
| (wt/v %) | (wt/v %) | (ml/min) | (mm) |
| 15 | 3 | 0.2 | <0.5 |
| | | 0.4 | 0.5-2.0 |
| | | 0.8 | 2.0-3.0 |
| | | 1.2 | Too fast |

When the flow rate was increased, the size of the beads increased. A higher flow rate at the dispersed phase delivered a larger volume of PCL/DCM solution for each formed droplet. This phenomenon resulted in larger macro-beads formed from PCL/DCM solutions of the same concentration.

### Effect of Concentration of PVA on Bead Size

PVA was used as the emulsifier. After preparing the macrospheres as described above, the macrospheres were rinsed with DI water several times to remove the PVA.

The hydroxyl groups in PVA interact with the water phase, while the polymer chain interacts with the dichloromethane, which makes the formed emulsion more stable. Variation in PVA concentration and volume can affect the emulsion stability, which can, in turn, affect the size of the macrospheres. As shown in Table 3, an increase in the PVA concentration led to a decrease in the size of the macrospheres.

TABLE 3
Effect of concentration of PVA on bead size

| PCL | Rate of | PVA | Size of | Type of beads |
|---|---|---|---|---|
| concentration | pump | concentration | beads | (depending on |
| (wt/v %) | (ml/min) | (wt/v %) | (mm) | the size) |
| 15 | 0.4 | 0.5 | 2.7-3.7 | Bead 1 |
| | | 1.0 | 1.8-2.2 | Bead 2 |
| | | 1.5 | 1.2-1.5 | Bead 3 |
| | | 2.0 | 0.5-1.0 | Bead 4 |
| | | 3.0 | 0.5-1.0 | Bead 4 |

When the concentration of PVA was increased, more PVA molecules overlay the surface of the droplets, providing increased protection of the droplets against coalescence which resulted in the production of smaller emulsion droplets. Since the macrospheres were formed from emulsion droplets after solvent evaporation, the size was dependent on the size of the emulsion droplets. Furthermore, the viscosity of the aqueous solution was higher at high PVA concentrations compared to lower concentrations, which may be another factor that contributes to the separation of droplets in the emulsion from each other.

### Characterizations and Measurements

Scanning electron microscopy (SEM) and Energy Dispersion Spectroscopy (EDS) analysis: the surface morphology of the PCL and PCL-PNIPAAm macro-carriers was observed by SEM (Carl Zeiss Evo LS15 VP-Scanning Electron Microscope SE, BSE, VPSE, EPSE detectors) at an accelerating voltage of 10 kV. Before the SEM investigation, the samples were coated with gold by sputtering. INCA X-Act X-ray system (Oxford Instruments), OIM XM 4 Hikari EBSD System (EDAX) for EDS analysis.

Fourier transform infrared (FTIR) spectra were recorded using the same equipment and method as described in Example 1.

### Size Distribution, Morphology and FITR of the PCL Macro-Beads

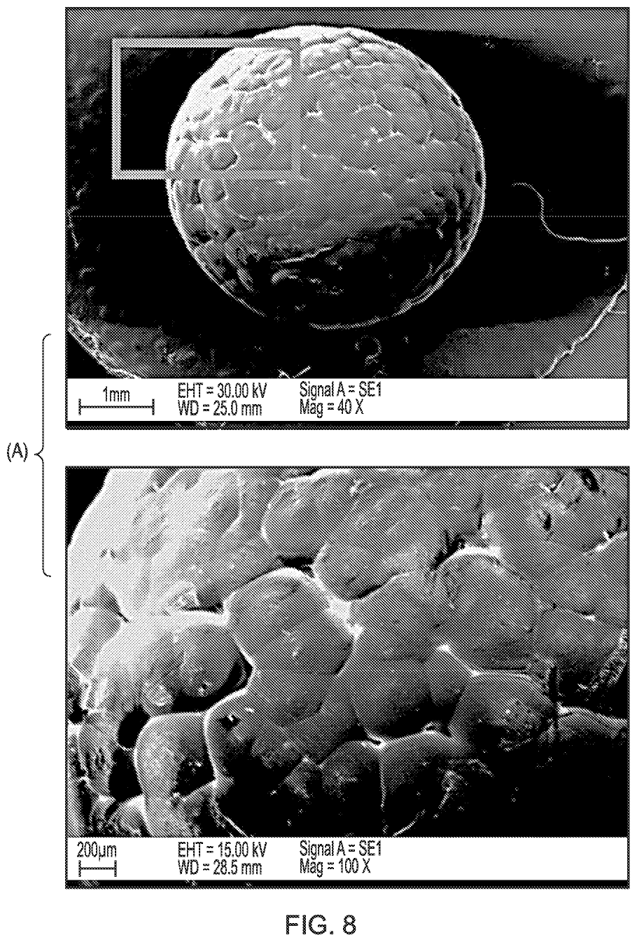

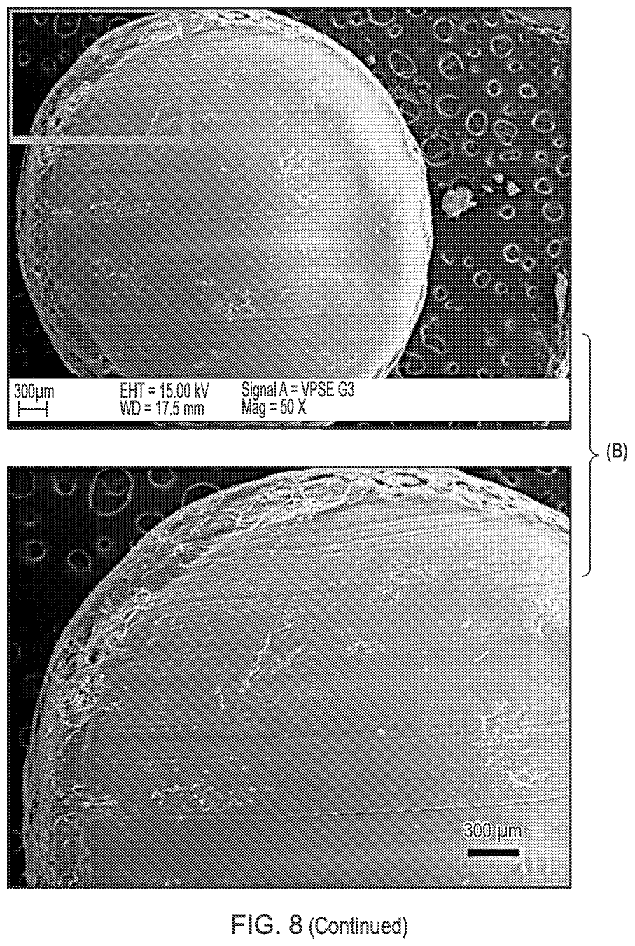

FIG. 8 shows the morphology of PCL macro-beads that were prepared using the method described above (see the images labelled (A), which are the “PCL-Oxford” beads) and those purchased from Sigma (see the images labelled (B), which are the “PCL-Sigma” beads). The surface of the bead shown in (A) was porous while the surface of the bead shown in (B) was non-porous and dense. The pores may be formed by the rapid precipitation of PCL, such as by using an organic solvent (e.g. DCM) having a low evaporation temperature during the solidification step. The pores can absorb and retain nutrients and medium on the surface of the beads. The porous surface can support cell adherence and growth better than a dense surface.

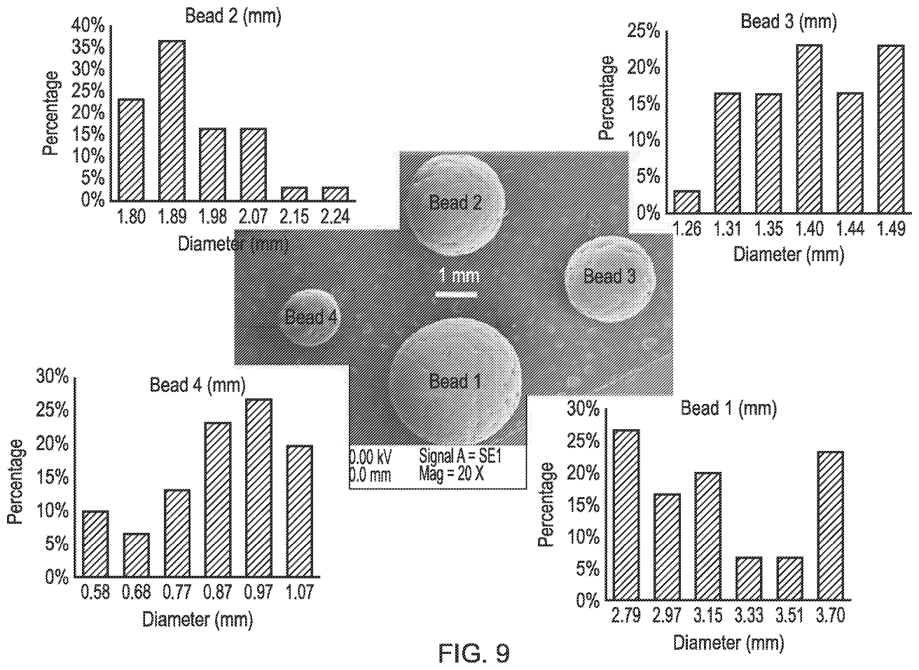

As shown in Table 3 above, four types of bead were prepared as described above at 15 wt/v% of PCL, rate of pump at 0.4 ml/min and various concentrations of PVA ranging from 0.5 to 3.0 wt/v%. The beads had diameters ranging from 0.5 to 3.7 mm. Bead 1 had diameters ranging from 2.7-3.7 mm, bead 2 had diameters ranging from 1.8-2.2 mm, bead 3 had diameters ranging from 1.2-1.5 mm, and bead 4 had diameters ranging from 0.5-1.0 mm. The morphology and size distribution of these beads are shown in FIG. 9. By controlling the PVA concentration and flow rate, the size of the beads could be controlled to obtain a uniform size. The average size of bead 1 was 3.09 mm, bead 2 was 1.89 mm, bead 3 was 1.37 mm and bead 4 was 0.83 mm.

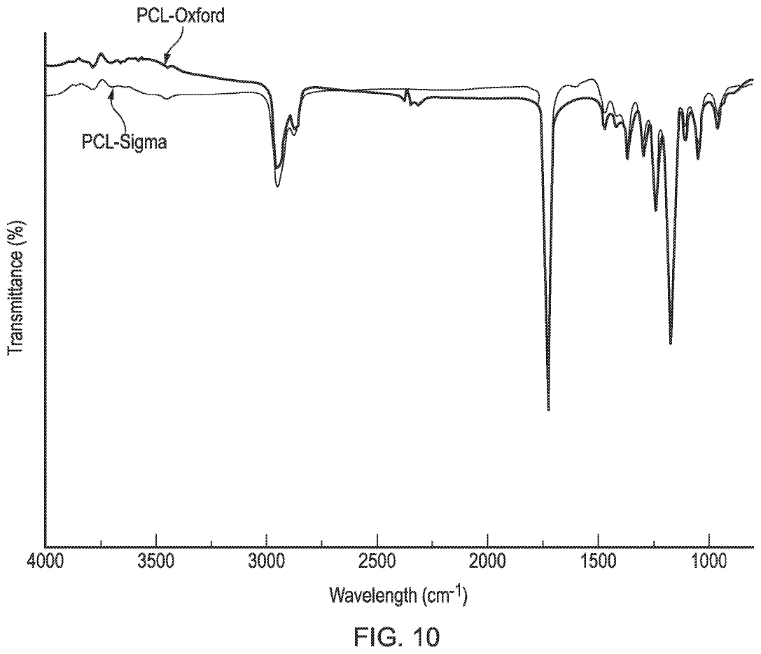

Results of the FITR spectra of PCL-Oxford and PCL-Sigma performed are shown in FIG. 10. These results showed that all the PCL-Oxford peaks matched all the PCL-Sigma peaks, which confirmed that the fabrication process did not change the chemical structure of PCL.

### Morphology of PCL and PCL-PNIPAAm Macro-Beads

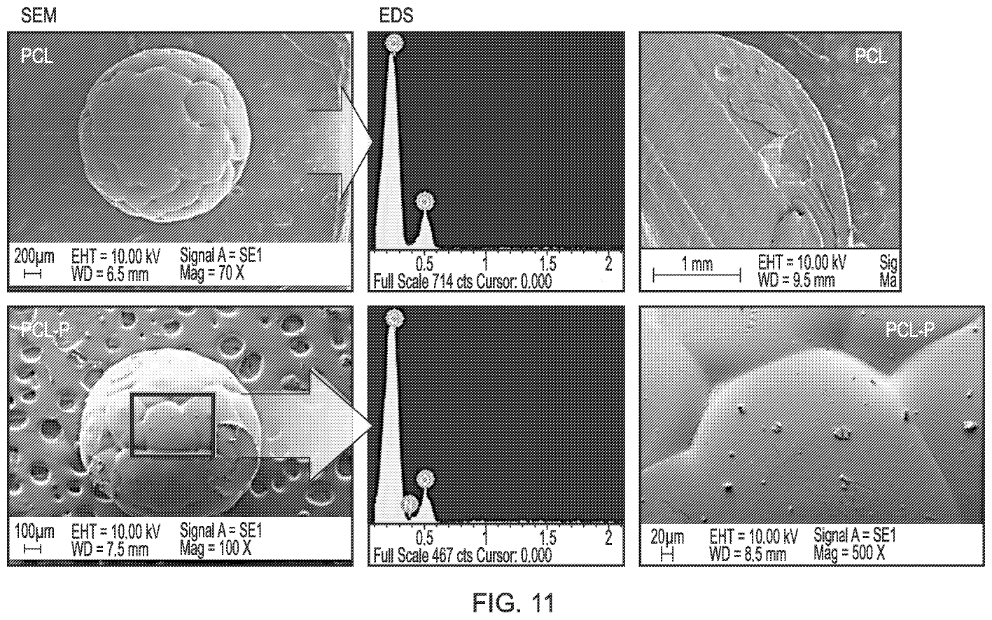

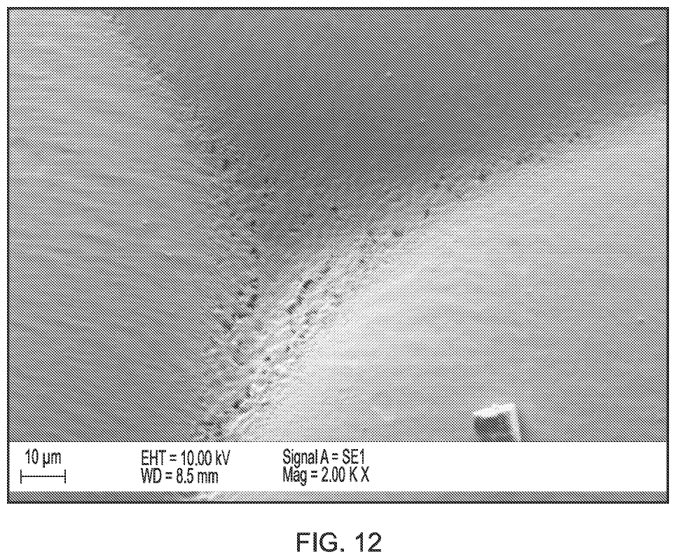

The porous PCL macro-beads (e.g. “PCL-Oxford” beads) were coated with PNIPAAm using the method described in Example 1. The surface morphology of the PCL and the resulting PCL-PNIPAAm macro-beads was characterized by SEM as shown in FIG. 11 (the PCL-PNIPAAm macro-beads are labelled “PCL-P” in the figure). The step of grafting PNIPAAm onto the porous PCL beads did not affect their surface morphology. This can be seen from FIG. 12, which shows a pore on the surface of a PCL-PNIPAAm macro-bead.

The appearance of a nitrogen peak in the EDS images (see FIG. 11) for the PCL-PNIPAAm (see between the C and O peaks in the EDS image for “PCL-P”) confirmed that the polymerization had been carried out properly. The surface morphology of the commercially available PCL beads (e.g. “PCL Sigma”) was unaffected by the presence of the PNIPAAm coating.

### Cell Culture on Macrocarriers

Green Fluorescence Protein (GFP) was cloned into Mesenchymal stem cells (MSC, provided by the Department of Paediatrics and Adolescent Medicine, LKS Faculty of Medicine, The University of Hong Kong). Briefly, primary mesenchymal cells (obtained from unfractionated bone marrow mononuclear cells of a healthy donor) were cultured for 2 months. Cells were infected with a VSV-G (expressing the G glycoprotein of the vesicular stomatitis virus) pseudotyped retroviral vector that contained the hTERT and green fluorescent protein (GFP) genes, separated by an internal ribosome entry site (IRES), under the control of the murine stem cell virus (MSCV) long-terminal repeat (LTR). The GFP+ and GFP-MSC then were separated with a fluorescence-activated cell sorter (MoFlo, Cytomation, Fort Collins, Colo., USA) 6. MSC-GFP were cultures in Dulbecco's modified Eagle's medium (DMEM 1.0 mg/l of glucose, Gibco BRL, Gaithersburg, Md., USA) supplemented with 10% (v/v) fetal bovine serum (FBS, Gibco BRL) and 0.1% (v/v) penicillin-streptomycin (PS, Gibco BRL).

The PCL and PCL-PNIPAAm macro-beads prepared above were placed in a laminar hood and UV radiation was applied for 30 minutes. Next, the PCL and PCL-PNIPAAm beads were immersed in 70% ethanol for 3 hours, washed with phosphate buffer saline (PBS) for 10 min and incubated in DMEM at 37° C., overnight. Cell seeding density was 2.8×10⁵ cells/ml.

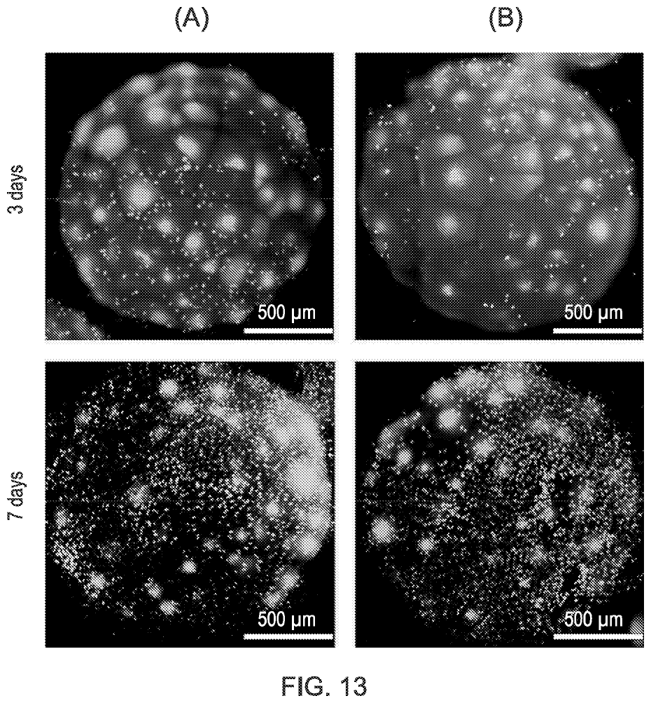

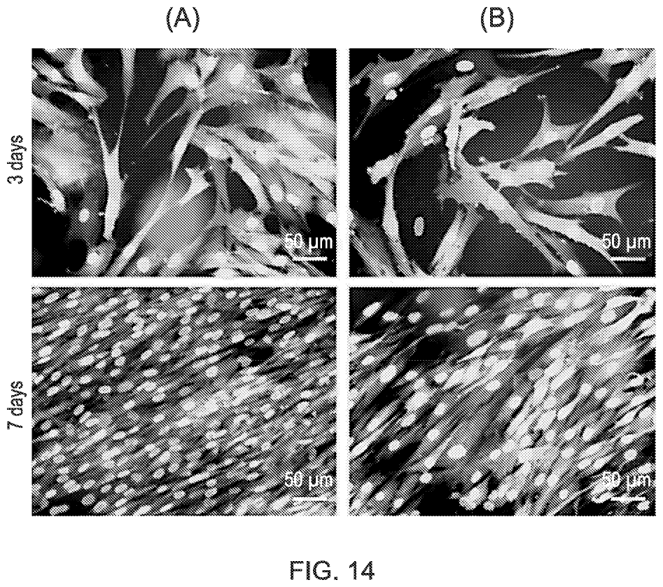

Cell proliferation of MSC seeded onto PCL and PCL-PNIPAAm after 3 and 7 days of incubation as determined by Hoechst staining and Green fluorescence protein (GFP) images are shown in FIGS. 13 and 14. FIG. 13 shows the cell proliferation of MSC seeded on PCL (see (A)) and PCL-PNIPAAm (see (B)) after 3 and 7 days, stained by Hoechst (Nuclei staining in blue dot), observed by fluorescence microscope in low magnification. FIG. 14 shows the cell proliferation of MSC seeded on PCL (see (A)) and PCL-PNIPAAm (see (B)) after 3 and 7 days, stained by Hoechst (Nuclei staining in blue dot) and green fluorescence protein, observed my fluorescence microscope in high magnification. The black background including the large blue dot areas are the beads.

The number of cells (the blue dot) on both PCL and PCL-PNIPAAm increased with increasing culture time from 3 days to 7 days. PCL used in this work had a molecular weight of 80 kDa (PCL80k). Very dense and clustered cells with higher viability were observed at day 7 and on grafted surfaces (PCL-PNIPAAm) than at day 3 and non-grafted PCL surfaces. Notably both groups of cells were healthy and showed their normal physiology and spindle shaped appearance. These results indicated that PNIPAAm grafting onto PCL surfaces was risk-free and can provide a valuable tool for recovering large-scale cellular collections.

### Cell Proliferation

### First Study

For cell viability and proliferation, the CCK-8 assay (Sigma) was performed after 1, 7, 14 and 21 days of incubation. Cell seeding density was 5×10³ cells/ml. For this experiment, around 20 g of PCL beads were prepared in the laboratory.

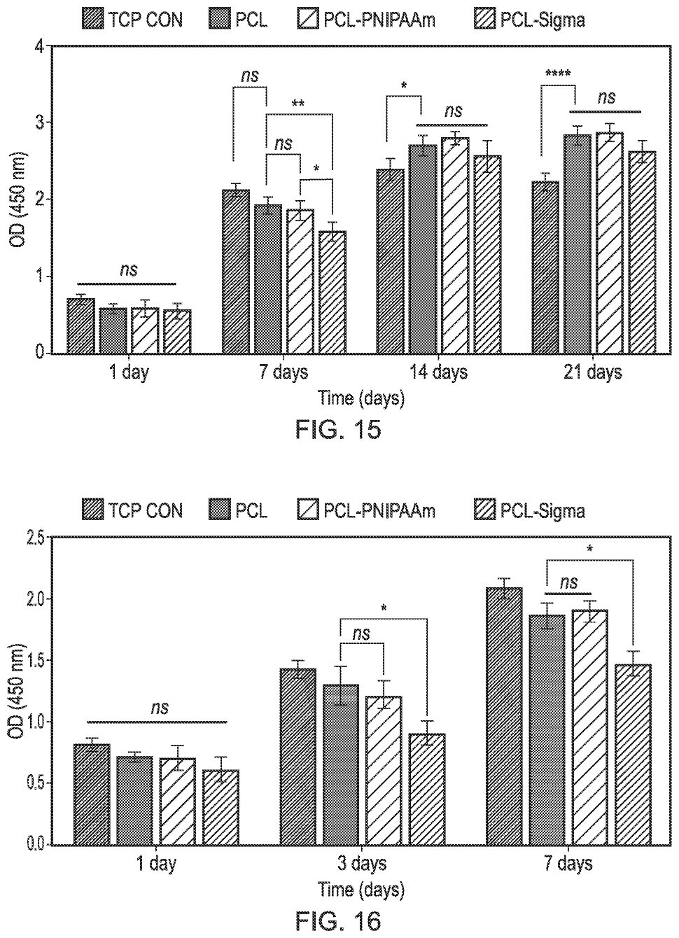

In this time dependent study, MSC proliferation was shown from 1 to 21 days on PCL-PNIPAAm and growth-proliferation was compared to different controls, which included on tissue culture plate (TCP), non-coated PCL beads and PCL beads from Sigma. The results are shown in FIG. 15.

At day 1, cells were found to be growing non-significantly on all sorts of surfaces. At day 7, cells proliferated significantly higher in TCP controls than with the PNIPAAm coated PCL surfaces and the commercially available PCL-beads (“PCL-Sigma”). There was no significant difference in proliferation between the cells on the non-coated PCL and the cells on the PCL-PNIPAAm samples. However, there was a slight significant difference in cell proliferation between (a) the non-coated PCL surfaces and the commercially available PCL-beads (p<0.01) and (b) the PCL-PNIPAAm samples and the commercially available PCL-beads (p<0.05).

At day 14, cells were found to be proliferating on all samples and controls. Proliferated cell numbers on non-coated PCL, PCL-PNIPAAm and the commercially available PCL-beads were measured to be non-significant. Cell proliferation on TCP was significantly lower than any other surface at day 14. On day 21, cell growth on non-coated PCL, PCL-PNIPAAm and PCL-Sigma was significantly high compared to cell proliferation on TCP.

Over the time-scale, an exponential proliferation of MSCs on all surfaces can be seen. At day 21, as compared to days 14 and 21, the cells were found to be in a stationary phase and a non-exponential growth pattern was observed. This is simply due to confluent growth of MSCs; there remains no room for any new cell growth in the culture.

### Second Study

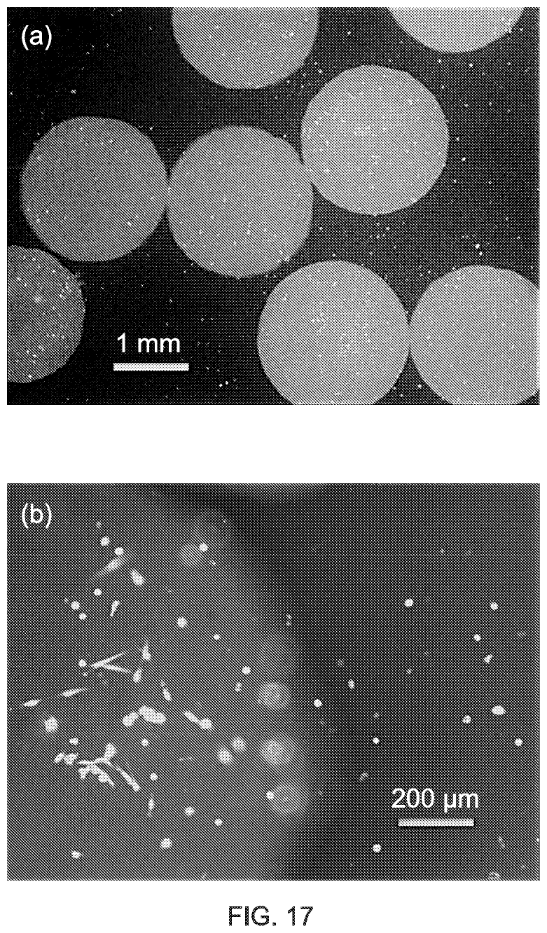

In this second study, the same procedure was used as in the first study except that the cell seeding density was 1×10⁴ cells/ml and the CCK-8 assay (Sigma) was performed after 1, 3 and 7 days of incubation. The results are shown in FIG. 16.

At day 3, the cells grown on the porous PCL and the porous PCL-PNIPAAm surfaces exhibited similar behaviour. However, a significant (p<0.0001) increase in OD was observed with porous PCL compared to the commercially available PCL-beads available PCL-beads (“PCL-Sigma”). The cell viability at day 7 had the same pattern as day 3, except that more cells survived on the surfaces of porous PCL beads than on the surfaces of the commercially available beads. No significant difference in cell viability for PCL and PCL-PNIPAAm surfaces was observed.

From the results, it can be seen that the cells grew better on the porous PCL beads than on the commercially available PCL-beads.

Cell Detachment from PCL-PNIPAAm

MSC detachment from PCL-PNIPAAm surfaces was observed under fluorescence microscopy. GFP loaded MSCs were grown on the sample surfaces overnight at 37° C. After this initial attachment, the incubation temperature was reduced from 37° C. to 25° C. for at least 1 hour. The detached cells from PCL-PNIPAAm were observed and imaged by an inverted microscope (Eclipse Ti, Nilon). Green fluorescence from the cells indicated free cells in the culture. FIG. 17 shows the MSCs detached from PCL-PNIPAAm at (a) low magnification and (b) high magnification.

1. A macrocarrier for the propagation of biological cells, said macrocarrier comprising substrate particles that are coated with a thermoresponsive polymer that is capable of providing the macrocarrier with a cell-receiving surface and responding to a change in temperature to release cells from the macrocarrier, wherein at least 50% of the substrate particles have a particle size of at least 1 mm.

2. A macrocarrier as claimed in claim 1, wherein the cell-receiving surface is porous.

3. A macrocarrier as claimed in claim 1, wherein at least 50% of the substrate particles have a particle size of 1 to 10 mm.

4. A macrocarrier as claimed in claim 3, wherein at least 50% of the substrate particles have a particle size of 2 to 6 mm.

5. A macrocarrier as claimed in claim 1, wherein at least 70% of the substrate particles have a particle size of 1 to 10 mm.

6. A macrocarrier as claimed in claim 5, wherein at least 70% of the substrate particles have a particle size of 2 to 6 mm.

7. A macrocarrier as claimed in claim 1, wherein at least 90% of the substrate particles have a particle size of 1 to 10 mm.

8. A macrocarrier as claimed in claim 1, wherein at least 90% of the substrate particles have a particle size of 2 to 6 mm.

9. A macrocarrier as claimed in claim 1, wherein the substrate particles are partially coated with the thermoresponsive polymer.

10. A macrocarrier as claimed in claim 1, wherein the particulate substrate comprises polycaprolactone.

11. A macrocarrier as claimed in claim 1, wherein the thermo-responsive polymer provides the macrocarrier with a cell-receiving surface above a threshold temperature and releases cells from the macrocarrier below the threshold temperature.

12. A macrocarrier as claimed in claim 11, wherein the threshold temperature is a temperature of 20 to 40 degrees C.

13. A macrocarrier as claimed in claim 1, wherein the thermoresponsive polymer is capable of transforming from a hydrophilic, cell-receiving state to a hydrophobic, cell-releasing state in response to a change in temperature.

14. A macrocarrier as claimed in claim 1, wherein the thermoresponsive polymer comprises poly(N-isopropyl acrylamide) (PNIPAAm).

15. A macrocarrier as claimed in claim 11, wherein the substrate particles comprise polycaprolactone and wherein the poly(N-isopropyl acrylamide) (PNIPAAm) is coupled to the polycaprolactone via an amide linkage.

16. A macrocarrier as claimed in claim 1, which further comprises biological cells attached to the thermoresponsive polymer.

17. A macrocarrier as claimed in claim 16, wherein the biological cells are selected from mesenchymal stem cells (“MSCs”), human dermal fibroblasts (“HDFs”), human umbilical vein endothelial cells (HUVEC) and neuroblastoma Sy5y cells.

18. A system for the propagation of biological cells, said system comprising a bio-reactor and a macrocarrier as claimed in claim 1.

19. A process for the propagation of biological cells, said process comprising:
a. contacting biological cells with a macrocarrier as claimed in any one of claim 1 in a cell culturing medium;
b. propagating the cells on the macrocarrier by subjecting the macrocarrier to a temperature at which the thermoresponsive polymer presents a cell-receiving surface; and
c. altering the temperature of the macrocarrier to release any propagated cells from the macrocarrier.

20. A process as claimed in claim 19, wherein the biological cells are contacted with the macrocarrier in a bioreactor, and wherein the macrocarrier is exposed to a first temperature of 20 to 40 degrees to propagate the cells, and wherein the temperature of the macrocarrier is reduced below the first temperature to release the propagated cells.

21. (canceled)
